(19) United States
(12) Patent Application Publication
(10) Pub. No.: US 2022/0386649 A1
(43) Pub. Date: Dec. 8, 2022

(54) COMPOSITION COMPRISING AMINO ACIDS AND A LIPID MATRIX FOR NON-MAMMALIAN MONOGASTRIC ANIMALS AND USE THEREOF

(71) Applicant: VETAGRO INTERNATIONAL S.R.L., Reggio Emilia (RE) (IT)

(72) Inventor: Andrea PIVA, Reggio Emilia (RE) (IT)

(21) Appl. No.: 17/631,400

(22) PCT Filed: Jul. 31, 2020
(86) PCT No.: PCT/IB2020/057268

§ 371 Date: Jan. 28, 2022

(30) Foreign Application Priority Data
Jul. 31, 2019 (IT) 102019000013449

(51) Int. Cl. A23K 20/142; A23K 20/158; A23K 40/10; A23K 50/80; A23K 20/28; A23K 20/24; A23K 50/75

(52) U.S. Cl. CPC A23K 20/142; A23K 20/158; A23K 40/10; A23K 50/80; A23K 20/28; A23K 20/24; A23K 50/75

(57) ABSTRACT

A composition for use in a method for the treatment of an amino acid deficiency to a non-mammalian monogastric animal, such as a poultry animal, a fish or a crustacean is described, wherein said composition comprises (i) a mixture of active components comprising or, alternatively, consisting of at least one amino acid or an analogue, (ii) a lipid matrix embedding said (i) mixture of active components and, optionally, (iii) at least one acceptable pharmaceutical or food grade additive and/or excipient. Furthermore, a feed or feed additive for a non-mammalian monogastric animal, such as poultry species and/or aquatic species comprising the composition is also described.

The present invention relates to a composition, preferably in solid form of granules, for use in a treatment method for the amino acid supply or treatment of amino acid deficiency to a non-mammalian monogastric animal, such as a poultry animal, a fish or a crustacean, wherein said composition comprises (i) a mixture of active components comprising or, alternatively, consisting of at least one amino acid (or an analogue) or at least one amino acid and a phytocompound derivative (botanical), (ii) a lipid matrix embedding said (i) mixture of active components and, optionally, (iii) at least one acceptable pharmaceutical or food grade additive and/or excipient. Furthermore, the present invention relates to the use of said composition, comprising (i) and (H) and, optionally, (iii), for the preparation of a feed or feed additive for a non-mammalian monogastric animal, preferably for poultry species and/or aquatic species. Lastly, the present invention relates to a feed or feed additive for a non-mammalian monogastric animal, preferably for poultry species and/or aquatic species, comprising or alternatively consisting of the composition of the invention.

Up to date, in order to meet the large market demand, the farming of non-mammalian monogastric animals, such as poultry and aquatic species, occurs on medium and large scale and requires consumption of a considerable amount of feed capable of ensuring an appropriate supply of amino acids to said animals, hence guaranteeing a healthy growth and appropriate development thereof.

The possibility of supplementing the diet with synthesis amino acids, i.e. amino acids of synthetic origin but identical to those present in natural sources of proteins, allows to finalise the fulfilment of nutritional requirements (limiting amino acids) without wasting proteins and at a low cost.

For example, according to literature, the protein levels commonly found in corn/soybean-based diets for broiler (23% of raw protein) contains various excess amino acids such as leucines (170% of intake from NRC (American national research council)), glycine and serine (157%), histidine (153%), phenylalanine, tyrosine, tryptophan, valine and arginine. Such excess amino acids are not used for metabolic purposes and they are excreted by the animal into the environment, causing negative environmental impact.

Furthermore, in the context of a widespread decrease of use of antibiotics in farms, excess protein can lead to an increased proliferation of Clostridi and coccidia, the main cause of necrotic enteritis, a serious problem in large-scale broiler farms worldwide.

As concerns aquatic species, protein is the most expensive part of the composition of the feed dedicated to this species and there is felt the need to find protein sources alternative to fishmeal.

Furthermore, feed or feed additives based on amino acids or derivatives thereof administered to aquatic species frequently suffer from leaching-related problems, given that part of the amino acids are dispersed into the aquatic environment.

In addition, in animals with an intestinal epithelium damaged by stress (for example, stress caused by intensive animal farming) and by inflammatory insults of various nature (such as, harmful ingredients present in the diet, pathogenic infections, environmental stress such as heat stress, etc.) the action of absorption of nutrients (for example amino acids) by the intestine is impaired, given that the intestinal epithelium is the area in which the enterocytes responsible for the absorption of nutrients reside.

Thus, the technical problem addressed and solved by the present invention lies in providing amino acid-based compositions, or feed or feed additives comprising said amino acid-based compositions, for an efficient and calibrated amino acid supply to non-mammalian monogastric animals (such as poultry animals, fish and crustaceans) capable of sustaining and/or increasing and/or accelerating the development of the body mass thereof and capable of treating amino acid deficiency, in the absence of the aforementioned negative effects of the prior art.

In particular, the technical problem addressed and solved by the present invention lies in providing an amino acid supply capable of decreasing the excretion of nitrogen by said animals and, thus, the environmental impact of the farms, capable of decreasing the presence of excess protein in the intestine of said animals and, thus, the possibility of bacterial infections, capable of decreasing the leaching of the amino acidic components into the aquatic environment and, furthermore, one that is cost-effective.

In addition, the technical problem addressed and solved by the present invention lies in providing an amino acid supply so that the blood bioavailability of the amino acids is constant within a period of time from 2 hours to 24 hours.

In addition, besides amino acids as essential elements for their growth and development, the technical problem addressed and solved by the present invention lies in providing said non-mammalian monogastric animals (such as poultry animals, fish and crustaceans), also with compounds capable of maintaining and/or restoring the integrity and homeostasis of the intestinal epithelium, so as to ensure efficient and effective absorption of the amino acids, less waste of energy by the body, and an effective and rapid response by the immune system. The primary function of the intestine lies in an efficient digestion and absorption of nutrients; these functions are affected if there are stress conditions that damage the intestinal mucosa, creating malabsorption and/or limiting the development of an immune response in case of need. Thus, it is possible to find an inadequate or unbalanced amino acid bioavailability quality/quantity-wise in animal subjects having a damaged intestinal epithelium as a result of stresses induced at bowel level (such as bacterial infections, parasitic infections, environmental stresses, etc.)

Lastly, the technical problem addressed and solved by the present invention lies in providing gastroprotected amino acids (or amino acids and other active compounds) so as to be able to administer said amino acids (acidic substances) or amino acids together with said further active compounds through enteral route to non-mammalian animals with a stomach (such as poultry animals, fish and crustaceans) without damage to the walls of the gastric tract and/or without the degradation of the very amino acids and/or of the further active compounds in the strongly acidic environment of the stomach.

In order to meet the aforementioned needs, following an intense research and development stage, the Applicant found that embedding active components, such as amino acids or amino acids and phytocompound derivatives, in lipid matrices, as reported in the present description and in the claims, provides both a suitable gastroprotection of the active components in case of animals with a stomach (such as poultry animals, fish and crustaceans) as well as a controlled release over time of the active components into the intestine and/or into the hepatopancreas of said non-mammalian monogastric animals (such as poultry, fish and crustaceans) to which a composition comprising said active components and said lipid matrices (in short, composition of the invention) is administered through oral route.

Said gastroprotection and/or said controlled release over time of the active components in the intestine and/or hepatopancreas guarantee a high and constant blood bioavailability of the active components (such as, amino acids) over time, in particular from 2 hours to 24 hours after the administration of the composition.

The lipid matrix of the invention is stable at the predominantly acidic pH of the stomach or ventriculus in the case of poultry (pH 2-6, depending on the digestive stage) (see, FIG. 1 for fish), hence, by embedding or incorporating the amino acids and, if present, the other active components (for example phytocompound derivatives), the lipid matrix allows them to pass through the stomach without undergoing degradation. When the composition of the invention reaches the intestine and/or the hepatopancreas, where the pH has a higher value with respect to the stomach (pH 4-7) and where there are enzymes capable of digesting lipid substances (i.e. lipases), the lipid matrix dissolves slowly allowing a controlled release of the active components and a constant blood bioavailability of the amino acids within a period of time comprised in the range from 2 hours to 24 hours. The intestine and/or the hepatopancreas have an enzymatic system rich in lipases which, by digesting the lipid matrix, allow the controlled release of the active components. The hepatopancreas is an organ located between the stomach and the intestine present in many aquatic species, in particular crustaceans and prawns, where the same enzymatic system as the intestine (i.e. Lipases) is present. By disintegrating along the intestinal tract over a period of time comprised from 30 minutes to 8 hours, preferably 1 hour to 6 hours, said lipid matrix allows the amino acids and/or said phytocompound derivatives to be gradually released in the various portions of the gastrointestinal tract. A gradual release of said phytocompound derivatives in the various portions of the gastrointestinal tract enhances the efficacy of the antioxidant activity, anti-inflammatory and immunostimulant activities thereof and, thus, the efficacy thereof at maintaining or restoring the homeostasis and the health of the microbiota of the intestinal mucosa. In addition, said phytocompound derivatives described in the present invention show an antibacterial activity which contributes to intestinal health. Table 1 shows—regarding chicken—an example of the transit time of the feed upon variation of the pH in the various segments of the digestive tract. As regards prawns and crustaceans, the transit time of the feed in the intestinal tract is instead comprised from 30 minutes to 120 minutes, preferably from 40 minutes to 90 minutes.

This entails that the addition of suitable amino acids to the diet of non-mammalian monogastric animals (such as poultry animals, fish and crustaceans) by means of the composition of the invention allows to boost the efficiency of the administered amino acids and/or phytocompound derivatives and thus to decrease both the nitrogen excreted hence limiting the environmental impact produced by a farm, as well as the excess undigested protein in the intestine hence limiting the onset of potential infectious diseases, and to increase the intestinal absorption of the amino acids administered to the animals.

The presence of phytocompound derivatives in the composition of the invention guarantees the supply—to said non-mammalian monogastric animals (such as poultry animals, fish and crustaceans)—of substances with antioxidant, anti-inflammatory and/or immunostimulant properties for the maintenance and/or restoration of the integrity of the intestinal mucosa. Providing both an adequate supply of specific amino acids known for their trophic effect on the intestinal mucosa, and phytocompound derivatives capable of maintaining and/or restoring the state of health of the intestinal mucosa and treating the dysbiosis of the intestinal epithelium, helps to compensate for malabsorption of nutrients (for example amino acids) due to direct stress on the mucosa, and it helps the production of key proteins for an effective immune response.

Specifically, the main limiting amino acids (or analogues) in non-mammalian monogastric animals, in particular in the poultry species and in the aquatic species, are: methionine and lysine, followed by cysteine, valine, isoleucine, tryptophan, leucine, threonine, arginine, histidine, tyrosine, phenylalanine, followed by glutamine, proline, hydroxyproline, glycine and taurine.

Furthermore, when the composition of the invention is administered to non-mammalian monogastric animals of the aquatic species, the embedment of said active ingredients in lipid matrices according to the invention to the invention decreases the dispersion of the active components into the aquatic environment (i.e. leaching).

Meeting the intake of limiting amino acids through the composition of the invention helps to decrease the protein percentage in the diet leading to an economic advantage in terms of cost of feed.

Lastly, the compositions of the invention and the feeds comprising the compositions of the invention as defined in the present invention are devoid of adverse effects and easy to prepare.

### FIGURES

FIG. 1, gastric pH (mean±DS) during the 24-hour cycle in the young ones of Diplodus sargus (family of Teleostei). The same letter does not indicate any significant difference (P>0.05). Dark grey area (about 10.00 hrs): feeding time. Light grey area (about 21.00-8.00 hrs): dark period.

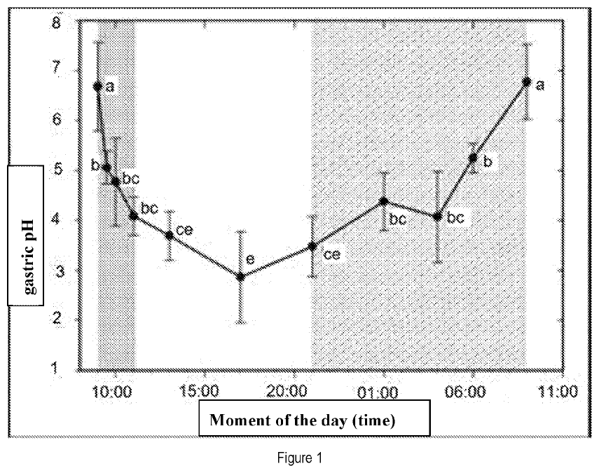

TABLE 1
Mean transit times at different pHs of the
segments of the digestive tract of broilers

| Segment of the digestive tract of broilers | pH | Transit time (min) |
|---|---|---|
| Gizzard | 5.5 | from 10 to 50 |
| Proventriculus/Ventriculus | 2.5-3.5 | from 30 to 90 |
| Duodenum | 5-6 | from 5 to 10 |
| Fasting | 6.5-7.0 | from 20 to 30 |
| Ileum | 7.0-7.5 | from 50 to 70 |
| Cecum/colon | 8.0 | from 20 to 30 |

### DETAILED DESCRIPTION OF THE INVENTION

Forming an object of the present invention is a composition (in short, composition of the invention) comprising or, alternatively, consisting of (i) at least one active component or a mixture of active components (in short, mixture of active components of the invention, according to a first embodiment (FR-I) or according to a second embodiment (FR-II)), (ii) a lipid matrix embedding or incorporating or dispersing said (i) and, optionally, (iii) at least one additive and/or excipient, as defined hereinafter.

Forming an object of the present invention is a composition (in short, composition of the invention) comprising or, alternatively, consisting of (i) at least one active component or a mixture of active components (in short, mixture of active components of the invention, according to a first embodiment or a second embodiment), (ii) a lipid matrix embedding or incorporating or dispersing said (i) and, optionally, (iii) at least one additive and/or excipient, as defined hereinafter, wherein said composition (according to any one of the embodiments of the present invention) is for use in a method for treating an amino acid supply or in a method for treating an amino acid deficiency in a non-mammalian monogastric animal, preferably animals of the poultry species and of the aquatic species (fish) and reptiles; even more preferably poultry animals, fish and crustaceans.

Forming an object of the present invention is the use of the composition of the invention comprising or, alternatively, consisting of (i) at least one active component or a mixture of active components, (ii) a lipid matrix embedding or incorporating or dispersing said (i) and, optionally, (iii) at least one additive and/or excipient (according to any one of the embodiments of the present invention) for preparing a feed or feed additive for a non-mammalian monogastric animal (in short, feed of the invention).

Furthermore, forming an object of the present invention is a feed or feed additive for a non-mammalian monogastric animal (in short, feed of the invention) comprises or, alternatively consists of the composition of the invention comprising or, alternatively, consisting of (i) at least one active component or a mixture of active components, (ii) a lipid matrix embedding or incorporating or dispersing said (i) and, optionally, (iii) at least one additive and/or excipient, according to any one of the embodiments of the present invention.

Lastly, forming an object of the present invention is a composition of the invention, comprising (i) and (ii) and, optionally, (iii) (according to any one of the embodiments of the invention), obtained/obtainable according to the preparation process of the present invention (step (I), (II) and (III)) described hereinafter.

In the context of the present invention, the expression “a non-mammalian monogastric animal” is preferably used to indicate an animal of the poultry species (class of Ayes, preferably the order of Galliformes), such as chicken or other fowls, turkey, guinea fowl, pheasant, peacock, partridge, quail, pigeon, turtle dove, goose, domestic duck and muscovy duck (preferably, chicken, laying hen and turkey), or, alternatively, an animal belonging to the aquatic species, such as fish and crustaceans (preferably farmed fish, for example salmon, trout, bass, gilthead bream, tilapia, carp, black bullhead and the like, and farmed crustaceans, for example prawns), or, alternatively, an animal belonging to the class of reptiles (class of Reptilia or simply reptile).

In the context of the present invention, the term “amino acids” refers to L- or D-α-amino acids, i.e. those whose amino group and carboxylic group are bound to the same carbon atom, called carbon a, in L or D configuration, thus having relative optical activity, with the sole exception of glycine, which is achiral. For example, in the case of methionine L-, D- or L-D-methionine mixture is available. Amino acids are the constitutive units of proteins (proteinogenic); a huge number of proteins can be obtained depending on the type, number and sequence order with which the different amino acids bind. In nature, we are generally familiar with 20 proteinogenic amino acids. The organism of a monogastric subject can synthesise some of the amino acids required to build proteins, but it is not capable of building others, which are therefore called “essential amino acids” (EAAs) and must be introduced through food.

In the context of the present invention, according to a first definition, the term “bioavailability” is used to indicate the “absolute bioavailability”, such as a fraction of compound under analysis in the systemic circulation following non-intravenous (e.g. oral) administration. Absolute bioavailability compares the bioavailability of a compound in the systemic circulation following non-intravenous (e.g. oral) administration with the bioavailability of the same compound following intravenous administration. It is therefore the fraction of compound absorbed through non-intravenous administration compared with that of the corresponding intravenous administration. The comparison should be normalised with respect to the dose, therefore the absorbed amount is corrected by dividing by the corresponding administered dose. The absolute bioavailability for a compound administered through oral route (po) is obtained from the oral area under the curve (AUCₚₒ) corrected by dose (D) divided by the intravenous area under the curve (AUCᵢᵥ) [F_{abs}=100·(AUCₚₒ·Dᵢᵥ)/(AUCᵢᵥ·Dₚₒ) %]. Said bioavailability can be measured by means of methods and apparatus known to the man skilled in the art, for example according to the methodology reported hereinafter in the present experimental part.

According to a second definition, in the context of the present invention, the term “bioavailability” is used to indicate the “relative bioavailability”, such as fraction of a compound under analysis (e.g. compound according to the invention) in the systemic circulation following the oral administration thereof in comparison with the fraction of a comparison compound (e.g. a feed or a composition not according to the invention) in the systemic circulation following the oral administration thereof. Said relative bioavailability of the compound under analysis can be expressed as a percentage considering 100% the fraction absorbed in the blood of the comparison compound: in this case, the percentage expressing the relative bioavailability of the compound under analysis may be less than 100% (lower bioavailability with respect to the comparison compound) or higher than 100% (higher bioavailability with respect to the comparison compound). Alternatively, said relative bioavailability of the compound under analysis can be expressed as a percentage difference with respect to the 1 (or 100) value of the blood-absorbed fraction of the comparison compound. For example, the following method may be used to determine the bioavailability of a composition according to the invention comprising lysine and a phytocompound derivative and a lipid matrix (e.g. rapeseed oil): two animal study groups are prepared, group 1 is administered with 1 kg of feed containing 40% of proteins, of which said proteins contain 10% of lysine (1 kg feed=40 g of lysine); group 2 is administered with an amount of composition of the invention containing 40 g of lysine. At a set time, the blood is collected from the animals of group 1 and group 2 and the mean lysine value present in the blood (in short, amount of lysine) is determined (for example by HPLC-MS) for each group. The amount of lysine determined for group 1 is set as a value 1 or a value of 100%, the amount of lysine determined for group 2 is expressed as a percentage or percentage difference with reference to said value 1 or 100%. Thus, if the amount of lysine in the blood of the Group 2 animals is, for example, 1.2 μg/ml and the amount of lysine in the blood of the Group 1 animals is 1.0 μg/ml, the bioavailability (relative bioavailability) of lysine of the composition of the invention is 120% or 20% more with respect to the bioavailability of lysine administered through the feed.

Preferably, in the context of the present invention the bioavailability data are expressed as relative bioavailability given that quantification of amino acid levels in the blood by means of the absolute bioavailability method is difficult to apply.

The composition of the invention, comprising said (i) at least one active component or mixture of active components embedded or incorporated or dispersed in said (ii) lipid matrix (both according to FR-I and according to FR-II), is obtained through the preparation process described in the patent application no EP 1 391 155 A1 paragraphs [0048]-[0049] and [0077]; said paragraphs are incorporated in the present description for reference. In short, said preparation process of comprises the steps of:
(I) preparing said (ii) lipid matrix according to the embodiments described in the present invention and, if present, said (iii) at least one additive and/or excipient to obtain a homogeneous mass (I) (step (I), T about 80-120° C.), followed by
(II) dispersing in said homogeneous mass (I) said (i) mixture of active components, according to the embodiments described in the present invention, to obtain a mass (II) (step (II), T about 55°−70° C.), followed by
(Ill) spraying the mass (II) in a cold room (step (III), T lower than 15° C.), to obtain the composition of the invention in form of particles, preferably relatively spherical-shaped, wherein each particle comprises said (i) at least one active component or a mixture of active components and, if present, said (iii) mixed additives and/or excipients are dispersed or incorporated or embedded in the (ii) lipid matrix. In other words, the composition of the invention is aggregate of (i), (ii) and, if present, (iii), wherein (i) is dispersed in (ii).

Thus, in the context of the present invention, the expression “(ii) a lipid matrix embedding or incorporating or dispersing said (i) at least one active component or a mixture of active components” is used to indicate an aggregate of the active components, such as amino acids and phytocompound derivatives embedded in said (H) lipid matrix.

The expression “(ii) lipid matrix embedding or incorporating or dispersing said (i) at least one active component or a mixture of active components” does not identify active components, such as amino acids or amino acids and phytocompound derivatives, coated with a film of said (ii) lipid matrix.

Furthermore, the expression “(ii) lipid matrix embedding or incorporating or dispersing said (i) at least one active component or a mixture of active components” does not identify active components, such as amino acids or amino acids and phytocompound derivatives, in the form of tablets, pills or analogue forms coated with said (ii) controlled release lipid matrix or with film of said (ii).

The advantages mentioned in the present invention of the composition of the invention and of the feeds of the invention stem from the chemical/physical properties of said (H) lipid matrix (for example, lipophilicity, resistance to disintegration at acidic pH, speed of disintegration at neutral or slightly acidic pH and in the presence of enzymes), as well as from the specific embedment or dispersion or incorporation of said (i) at least one active component or a mixture of active components in the (ii) lipid matrix obtained through the preparation method described above.

In the composition of the present invention, following an oral administration of said composition of the invention to an animal said (ii) lipid matrix facilitates a gastroprotection of the mixture of active components in the case of species with stomach and a controlled release of said (i) one active component or mixture of active components in the intestine and/or in the hepatopancreas, guaranteeing a constant blood bioavailability of said (i) one active component or mixture of active components within a period of time comprised in the range from 2 hours to 24 hours. Furthermore, said (ii) lipid matrix decreases the dispersion of active components in the aquatic environment (i.e. leaching).

The term “bioavailability of a constant amino acid” is used to indicate a constant value of relative bioavailability (with respect to the bioavailability of the same amino acid provided by a feed or a comparison compound) comprised from 101% to 200%, preferably from 101% to 150%, more preferably from 101% to 125%, equivalent to a positive percentage difference (greater bioavailability of the amino acid provided by the compound of the invention with respect to the feed or comparison) comprised from 1% to 100%, preferably from 1% to 50%, more preferably from 1% to 25% (for example 2%, 4%, 5%, 6%, 8%, 10%, 15%, 20%).

Furthermore, the advantages of the composition of the invention mentioned in the present invention (FR-I and FR-II) derive from the particle size of the composition of the invention (or particle size percentage distribution), and from the characteristic of the granules of the composition of the invention to disintegrate at different times and in different sections of the intestine as a function of the particle size thereof.

Advantageously, the composition of the invention and the feed or feed additive comprising the composition of the invention have the properties of increasing and/or accelerating the development of the body mass, decreasing the excretion of nitrogenated compounds and decreasing the excess of undigested proteins in the intestine and/or hepatopancreas of said non-mammalian monogastric animal.

Advantageously, the composition of the invention and the feed or feed additive comprising the composition of the invention have the property of preserving the integrity and the homeostasis of the intestinal mucosa and facilitating the restoration of the integrity thereof in the presence of a damage (for example, damage to the intestinal mucosa caused by stress or by an inflammatory insult).

According to a first embodiment of the present invention (in short, FR-I), the composition of the invention comprises (i), (ii) and, optionally, (iii), wherein

(i) is at least one active component or a mixture of active components comprising or, alternatively, consisting of (i.1) at least one amino acid or an analogue, or an acceptable pharmaceutical or food grade salt thereof; (ii) is a lipid matrix comprising or, alternatively, consisting of (ii.1) at least one saturated or unsaturated, free or esterified fatty acid, having a number of carbon atoms comprised in the range C10-C30, preferably C14-C24, and/or (ii.2) at least one triglyceride having saturated or unsaturated fatty acid chains, having a number of carbon atoms comprised in the range C6-C30, preferably C14-C24, and/or (ii.3) at least one wax having a number of carbon atoms comprised in the range C16-C36, preferably 024-C36; and (iii) is at least one acceptable pharmaceutical or food grade additive and/or excipient.

In the composition of the invention comprising said (i) and (ii) and, optionally, (iii), said (i) at least one active component o is at least one amino acid, or an analogue or a salt thereof, and it is selected from the group A comprising or, alternatively, consisting of: methionine, lysine, valine, isoleucine, tryptophan, histidine, leucine, phenylalanine, threonine, arginine, cysteine, tyrosine, glutamine, proline, hydroxyproline, glycine, taurine and mixtures thereof; preferably methionine, lysine and threonine.

In the composition of the present invention, the expression “analogue” of an amino acid is used to indicate a compound having—analogously to an amino acid—an amine or basic group and an acidic group, such as for example taurine. Taurine or 2-aminoethanesulfonic acid) is an amine with sulfonic acid functional group abundant in many tissues of various animals.

According to an aspect of said first embodiment of the invention (FR-I), in the composition of the invention comprising (i), (ii) and optionally, (iii), said at least one amino acid is a mixture of amino acids selected from the group B of mixtures comprising or, alternatively, consisting of: methionine and lysine; methionine and threonine; lysine and threonine; methionine and glutamine; lysine and glutamine; threonine and glutamine; tryptophan and glutamine; valine and glutamine; leucine and glutamine; isoleucine and glutamine; methionine, lysine and threonine; methionine, lysine and cysteine; valine, isoleucine and tryptophan; valine, isoleucine and threonine; methionine, valine, isoleucine and tryptophan; methionine, lysine, valine, isoleucine and tryptophan methionine, lysine, cysteine and valine; methionine, lysine, cysteine and isoleucine; methionine, lysine, cysteine, tryptophan; methionine, lysine, cysteine, valine and isoleucine; methionine, lysine, cysteine, valine and tryptophan; methionine, lysine, cysteine, isoleucine and tryptophan; methionine, lysine, cysteine, valine, isoleucine and tryptophan; leucine, valine and isoleucine; methionine, leucine, valine and isoleucine; methionine, lysine, leucine, valine and isoleucine; methionine, lysine, cysteine, leucine, valine and isoleucine; preferably methionine and lysine; methionine, lysine and threonine; methionine, lysine and cysteine; leucine, valine and isoleucine; valine, isoleucine and tryptophan.

Preferably, i said mixtures of 2 amino acids selected from said group B, such as: methionine and lysine; methionine and threonine; lysine and threonine; methionine and glutamine; lysine and glutamine; threonine and glutamine; tryptophan and glutamine; valine and glutamine; leucine and glutamine; isoleucine and glutamine, the two amino acids are at a by weight ratio with respect to each other comprised in the range from 1:10 to 10:1, preferably from 1:5 to 5:1, more preferably from 1:3 to 3:1, even more preferably 1:1.

According to a further aspect of said first embodiment (FR-I) of the present invention, in the composition of the invention comprising (i), (ii) and, optionally, (iii), besides said at least one amino acid or mixture of amino acids selected from group A and B described above, said (i) mixture of active components further comprises at least one first further active component selected from the group C comprising or, alternatively, consisting of: a non-proteinogenic amino acid, preferably selected from the group comprising or, alternatively, consisting of ornithine and alpha-ketoglutaric acid (or 2-oxopentanedioic acid); a protease (or proteolytic enzymes), preferably a cysteine-protease selected from the group comprising or, alternatively, consisting of bromelain, papain, ficain and mixtures thereof; a polyamine, such as trophic nutrient, selected from the group comprising or, alternatively, consisting of putrescine, spermine and spermidine; and mixtures thereof. Preferred examples are: methionine combined with ornithine and/or alpha-ketoglutaric acid and/or a protease and/or polyamine; lysine combined with ornithine and/or alpha-ketoglutaric acid and/or a protease and/or polyamine; threonine combined with ornithine and/or alpha-ketoglutaric acid and/or a protease and/or polyamine; tryptophan combined with ornithine and/or alpha-ketoglutaric acid and/or a protease and/or polyamine; leucine combined with ornithine and/or alpha-ketoglutaric acid and/or a protease and/or polyamine; valine combined with ornithine and/or alpha-ketoglutaric acid and/or a protease and/or polyamine; isoleucine combined with ornithine and/or alpha-ketoglutaric acid and/or a protease and/or polyamine; glutamine combined with ornithine and/or alpha-ketoglutaric acid and/or a protease and/or polyamine; or, alternatively, 2 or 3 or 4 or 5 or 6 amino acids, selected from any possible combinations of methionine, lysine, threonine, tryptophan, leucine, valine, isoleucine and glutamine, preferably a mixture belonging to group B, combined with ornithine and/or alpha-ketoglutaric acid and/or a protease and/or polyamine.

preferably, in said compositions comprising an amino acid or a mixture of amino acids selected from said group A or B, preferably methionine; lysine; threonine; methionine and lysine; methionine and threonine; lysine and threonine; methionine and glutamine; lysine and glutamine; threonine and glutamine; tryptophan and glutamine; valine and glutamine; leucine and glutamine; isoleucine and glutamine; methionine, lysine and threonine; methionine, lysine and cysteine; valine, isoleucine and tryptophan; valine, isoleucine and threonine; methionine, valine, isoleucine and tryptophan; methionine, lysine, valine, isoleucine and tryptophan methionine, lysine, cysteine and valine; methionine, lysine, cysteine and isoleucine; methionine, lysine, cysteine, tryptophan; methionine, lysine, cysteine, valine and isoleucine; methionine, lysine, cysteine, valine and tryptophan; methionine, lysine, cysteine, isoleucine and tryptophan; methionine, lysine, cysteine, valine, isoleucine and tryptophan; leucine, valine and isoleucine; methionine, leucine, valine and isoleucine; methionine, lysine, leucine, valine and isoleucine; methionine, lysine, cysteine, leucine, valine and isoleucine; preferably methionine and lysine; methionine, lysine and threonine; methionine, lysine and cysteine; leucine, valine and isoleucine; valine, isoleucine and tryptophan; and 1 or 2 or 3 or 4 said first further active component selected from said group C, such as: ornithine, alpha-ketoglutaric acid, a protease and polyamine, the components of group A or B and the components of group C are at an ((A o B):C) by weight ratio with respect to each other comprised in the from 1:10 to 10:1, preferably from 1:5 to 5:1, more preferably from 1:3 to 3:1, even more preferably 1:1.

In the composition of the invention, the optional presence of Krebs cycle intermediates, such as alpha-ketoglutarate or ornithine, facilitate the disposal of ammonia hence stimulating the urea cycle in the ureotelic species, or, alternatively, they facilitate the conversion of ammonia into amino acids and proteins (e.g. by stimulating the mTOR pathway, which, if activated, stimulates the protein synthesis) in the ammonotelic species, such as many aquatic species, or in the uricotelic species, such as poultry.

Proteases (or proteolytic enzymes) are enzymes that enhance protein digestion at intestinal level. The optional presence of protease in the composition of the invention allows to stimulate the digestion of protein portions that arrive undigested in the intestine, contributing toward increasing the amino acid pool at the intestinal level and, simultaneously, preventing the undigested protein portions from being used in the intestine as a substrate by bacterial pathogens.

In a further aspect of said first embodiment (FR-I) of the present invention, in the composition of the invention comprising (i), (ii) and, optionally, (iii), besides said at least one amino acid or mixture of amino acids selected from the group A and B described above and, optionally, besides said at least one first further active component selected from the group C described above said (i) mixture of active components, comprises at least one second further active component selected from the group D comprising or, alternatively, consisting of: at least one vitamin, preferably a vitamin of group B, at least one organic or inorganic acid, at least one mineral salt, at least one antioxidant, at least one probiotic bacterial strain, at least one prebiotic, at least one enzyme and mixtures thereof.

Advantageously, said vitamin is a vitamin of group A, B, C, D, E or K; preferably a vitamin of group B selected from the group comprising or, alternatively, consisting of B1, B2, B3, B4, B5, B6, B7, B8, B9, B10, B11, B12 and mixtures thereof.

Advantageously, said mineral salt is an organic or inorganic salt of a metal cation, such as, for example, Fe, Se, Mg, Ca, K, Zn and Cu.

Advantageously, said antioxidant is selected from N-acetyl cysteine (NAC), Coenzyme Q10 (CoQ10), acetyl-L-carnitine, and analogues.

Preferred examples of said compositions of the invention, comprising said (i) mixture of active components selected from groups A and/or B and C and D and said (ii) lipid matrix, comprise: an amino acid selected from methionine, lysine, threonine, tryptophan, leucine, valine, isoleucine and glutamine combined with ornithine and/or alpha-ketoglutaric acid and/or a protease and/or a polyamine combined with a vitamin (vitamin B) and/or an organic or inorganic acid and/or a mineral salt and/or an antioxidant and/or a probiotic bacterial strain and/or a prebiotic and/or an enzyme; or, alternatively, 2 or 3 or 4 or 5 or 6 amino acid, selected from any of the possible mixtures of methionine, lysine, threonine, tryptophan, leucine, valine, isoleucine and glutamine, preferably a mixture belonging to group B, combined with ornithine and/or alpha-ketoglutaric acid and/or a protease and/or a polyamine, and combined with a vitamin (vitamin B) and/or an organic or inorganic acid and/or a mineral salt and/or an antioxidant and/or a probiotic bacterial strain and/or a prebiotic and/or an enzyme.

Preferably, the components of group A or B and the components of group C and the components of group D are at a by weight ratio with respect to each other ((A o B):C:D) comprised in the range from 1:10:10 to 10:10:1 or 10:1:10, preferably from 1:5:5 to 5:5:1 or 5:1:5, more preferably from 1:3:3 to 3:3:1 or 3:1:3, even more preferably 1:1:1.

It should be observed that the components of group C and/or the components of group D can also be present in the composition of the invention according to the embodiment FR-II ((i) mixture of active components comprising (i.1) at least one amino acid and (i.2) at least one phytocompound derivative).

According to a second embodiment of the present invention (in short, FR-II), the composition of the invention is a composition, preferably a solid composition in the form of granules, comprising (i) a mixture of active components comprising, or alternatively, consisting of:
(i.1) at least one amino acid or an acceptable pharmaceutical or food grade salt thereof, wherein said amino acid is selected from a group comprising or, alternatively, consisting of: lysine (Lys), methionine (Met), tryptophan (Trp), threonine (Thr), valine (Val), isoleucine (Isoleu), arginine (Arg), histidine (His) and mixtures thereof;
(i.2) at least one phytocompound derivative (botanical) selected from a group comprising or, alternatively, consisting of: (a) thymol, (b) carvacrol, (c) eugenol, (d) capsaicin, (e) tannins, (f) verbascoside, (g) saponins and mixtures thereof; and

wherein said composition comprises (ii) a lipid matrix embedding said (i) mixture of active components, wherein said lipid matrix comprises or, alternatively, consists of: at least one saturated or unsaturated, free or esterified fatty acid having a number of carbon atoms comprised in the range C10-C30, and/or at least one triglyceride having saturated or unsaturated fatty acid chains, having a number of carbon atoms comprised in the range C6-C30 and/or at least one wax having a number of carbon atoms comprised in the range C16-C36; and optionally, said composition further comprises (iii) at least one acceptable food or pharmaceutical grade additive and/or excipient.

Said composition of the invention (according to said first embodiment (FR-I) or said second embodiment (FR-II)), preferably a solid composition in the form of granules is a composition for use in a method for the treatment of a deficiency of at least one amino acid in a non-mammalian monogastric animal, wherein said non-mammalian monogastric animal is a poultry animal or a fish or a crustacean; preferably wherein said poultry animal is selected from the group comprising or, alternatively, consisting of: chicken, laying hen and turkey, and/or said fish is a fish that can be farmed in fresh water or that can be farmed in salty water; even more preferably said fish is selected from the group comprising or, alternatively, consisting of: salmon, trout, bass, gilthead bream, tilapia, carp, black bullhead and the like, and farmed crustaceans such as for example prawns.

When said composition of the invention, preferably in the form of granules is administered through oral route, said (ii) lipid matrix is capable of providing a gastroprotection of said (i.1) at least one amino acid and said (i.2) at least one phytocompound derivative, said (ii) lipid matrix is capable of providing a controlled release of said (i.1) at least one amino acid and of said (i.2) at least one phytocompound derivative within a time range comprised from 30 minutes to 6 hours in the intestinal tract, and said (ii) lipid matrix is capable of providing a blood bioavailability of said (i.1) at least one amino acid at a constant percentage (as defined in the context of the present invention), over a period of time comprised from 2 hours to 24 hours.

With reference to said (ii) lipid matrix, comprised in the composition of the invention together with (i) and, optionally, (iii) (according to said first embodiment (FR-I) and said second embodiment (FR-II)), said (ii.1) at least one fatty acid may be a hydrogenated or non-hydrogenated fatty acid of plant and/or animal origin, preferably it is a hydrogenated fatty acid of plant origin selected from group E comprising or, alternatively, consisting of palm oil, sunflower oil, corn oil, rapeseed oil, peanut oil, soybean oil, olive oil and mixtures thereof (preferably rapeseed oil, palm oil, soybean oil and mixtures thereof); said (ii.2) at least one triglyceride may be a hydrogenated or non-hydrogenated triglyceride of plant and/or animal origin, preferably a hydrogenated triglyceride of plant origin selected from the group E comprising or, alternatively, consisting of palm oil, sunflower oil, corn oil, rapeseed oil, peanut oil, soybean oil, olive oil and mixtures thereof (preferably rapeseed oil, palm oil, soybean oil and mixtures thereof (preferably rapeseed oil, palm oil, soybean oil and mixtures thereof); and, lastly, said (ii.3) at least one wax may be a wax of plant and/or animal origin, preferably of animal origin, more preferably beeswax.

According to a further aspect of the invention, said (ii.2) at least one triglyceride may be a hydrogenated triglyceride of animal origin and/or said fatty acid may be a hydrogenated fatty acid of animal origin, being selected from chicken fat, hydrogenated chicken fat, bovine tallow and lard.

For example, according to said embodiment FR-II, the composition of the invention is a composition, preferably a solid composition in the form of granules, comprising (i) a mixture of active components comprising, or alternatively, consisting of: said (a.1) at least one amino acid, or a salt thereof, selected from a group comprising, or alternatively, consisting of: lysine (Lys), methionine (Met), tryptophan (Trp), threonine (Thr), valine (Val), isoleucine (Iso-Leu), arginine (Arg), histidine (His) and mixtures thereof: and said (b.4) at least one phytocompound derivative (botanical) selected from a group comprising or, alternatively, consisting of: (a) thymol, (b) carvacrol, (c) eugenol, (d) capsaicin, (e) tannins, (f) verbascoside and mixtures thereof; and wherein said composition further comprises said (ii) lipid matrix selected from the group comprising or, alternatively, consisting of: palm oil, rapeseed oil, soybean oil and mixtures thereof (preferably soybean oil); and, optionally, said (iii) at least one additive and/or excipient (preferably coating additives).

Triglycerides (or triacylglycerols) are neutral esters of glycerol in which chains of three long chain fatty acids are present instead of the hydrogen atoms of the hydroxyl groups. The length of the fatty acid chains in the common triglycerides structures may be from 5 to 28 carbon atoms, but 17 and 19 are more common.

The term fatty acids (in short FAs) is mainly but not exclusively used to indicate long-chain aliphatic monocarboxylic acids with an even number of carbon atoms, without branching and acyclic (i.e., consisting of molecules that do not have ring-like closed chains). Fatty acids can be saturated (if their molecule has single C—C bonds only) or unsaturated (if they have double C═C bonds).

The term waxes is used to indicate to long-chain fatty acid esters with high molecular weight monohydric alcohols. Waxes may be of plant origin or animal origin (beeswax).

Rapeseed oil is a vegetable food oil produced from rape seed (Brassica napus, Brassica rapa, Brassica juncea) and from cultivars or mutant varieties. A rapeseed oil which can be used in the context of the invention has, for example, the following distribution of fatty acids: myristic acid <0.5%, palmitic acid 9-16%, stearic acid: 79-89%, oleic acid and its isomers <4%, linoleic acid and its isomers <1%, linolenic acid and its isomers <0.2%, arachidic acid <1%, behenic acid <1%, saturated fatty acids with carbon chain length less than C14: <0.1%.

Palm oil, or palm fruit oil and palm seed oil, are vegetable oils, mainly consisting of triglycerides with high concentrations of saturated fatty acids, obtained from oil palms, mainly Elaeis guineensis but also from Elaeis oleifera e Attalea manipa. A palm oil which can be used in the context of the invention has, for example, the following distribution of fatty acids: lauric acid <1%, myristic acid <2%, palmitic acid <47%, stearic acid <58%, oleic acid <3%, trans fatty acids <1%.

Soy oil (or soy bean oil) is obtained by extraction from soy beans through a particular process called crush using chemical solvents. Soybean oil mainly consists of triglycerides with the following typical distribution of fatty acids, as indicated in Codex Alimentarius: palmitic acid 8.0-13.5%, stearic acid 2.0-5.4%, oleic acid 17-30%, linoleic acid 48.0-59.0%, α-linolenic acid 4.5-11.0%, and others up to 100%.

According to an aspect both of said first embodiment (FR-I) and said second embodiment of the invention (FR-II), the composition of the invention comprising (i), (ii) and, optionally, (iii), comprises—at a % by weight with respect to the total weight of the composition—said (i) at least one active component or mixture of active components, in the various embodiments defined in the present invention, in the range from 1% to 90% (for example 5%, 20%, 25%, 30%, 35%, 40%, 50%, 55% or 65%), preferably from 10% to 60% or from 5% to 50%, more preferably from 15% to 45%, and said (ii) lipid matrix, in the various embodiments defined in the present invention, in the range from 10% to 80% (for example 15%, 20%, 25%, 35%, 50% or 65%); preferably from 40% to 60% or from 30% to 70%, more preferably from 45% to 55%.

According to an aspect of said second embodiment of the invention (FR-II, for example from FR-II-1 to FR-II-10), the composition of the invention comprises: said (i.1) at least one amino acid from 1% to 80% (for example 5%, 10%, 15%, 20%, 25% or 30%), preferably from 5% to 40%, more preferably from 5% to 35%, said (i.2) at least one phytocompound derivative from 0.5% to 15% (for example 5%, 10%, 15%, 20%, 25% or 30%), preferably from 1% to 10%, more preferably from 1% to 5%, said (H) lipid matrix from 10% to 80% (for example 15%, 20%, 25%, 35%, 50% or 65%); preferably from 30% to 70%, more preferably from 45% to 55%, and, optionally, said (iii) additive and/or excipient (preferably said coating additive) from 0.1% to 30% (for example 0.5%, 2%, 4%, 6%, 8%, 15% or 25%), preferably from 1% to 20%, more preferably from 5% to 10%; wherein said percentages are percentages by weight with respect to the total weight of the composition.

Advantageously, the composition of the invention comprising (i), (H) and, optionally, (iii), is in solid form (granules or powder, preferably granules).

According to an aspect both of said first embodiment (FR-I) and said second embodiment of the invention (FR-II), the composition of the invention comprising (i), (ii) and, optionally, (iii), is in form of relatively spherical particles (synonyms: granules or microcapsules) having an average particle size (or particle size) comprised in the range from 50 μm to 2500 μm or 100 μm to 2000 μm (for example, 250 μm, 400 μm, or 500 μm), preferably from 200 μm to 1500 μm, more preferably from 250 μm to 500 μm. In particular, when the composition of the invention is intended for aquatic species (for example fish), said average particle diameter is comprised the range from 100 μm to 1000 μm, preferably from 250 μm to 500 μm. On the other hand, when the composition of the invention is intended poultry species, said average particle diameter is preferably in the range of from 250 μm to 2000 μm, more preferably from 500 μm to 1500 μm or from 500 μm to 1000 μm.

Within a batch of composition of the invention (according to FR-I or FR-II) in the form of granules, the granules do not all have the same particle size, but they have a particle size distribution percentage within the above indicated particle size ranges. Said particle size distribution percentage may vary depending on whether the composition is for use in the treatment of an amino acid deficiency in poultry animals or fish.

For animals of the aquatic species (fish or crustaceans) a batch of 100 granules of the composition may have the following particle size distribution percentage: from 10% to 20% of granules has a particle size from 50 μm to 250 μm, from 45% to 55% the particle size measures 250 μm-400 μm, from 20% to 30% the particle size measures 400 μm-500 μm, from 5% to 15% the particle size measures 500 μm-2500 μm (wherein said percentages are percentages of granules with respect to 100 granules).

For animals of the poultry species (e.g. chicken, laying hens, turkeys), a batch of 100 granules of the composition may have the following particle size distribution percentage: from 1% to 10% of granules has a particle size from 50 μm to 500 μm, from 45% to 55% the particle size measures 500 μm-1000 μm, from 35% to 45% the particle size measures 1000 μm-1500 μm, from 1% to 9% the particle size measures 1500 μm-2000 μm, from 0.1% to 1% the particle size measures 2000 μm-2500 μm (wherein said percentages are percentages of granules with respect to 100 granules).

Examples of batches of the composition of the invention (according to FR-I or FR-II) in the form of granules are reported in Table A. Said particle size percentage distribution is constant and reproducible in the preparation of the batches of the composition of the invention. Upon reaching the intestine, said granules break up at different times and in different sections of the intestine depending on their granulometry. Thus, the effect of said particle size distribution percentage consists in a slow and progressive release of the active compounds comprised in the mixture M embedded in the lipid matrix along the whole section of the intestine. In particular, the smaller granules are digested (releasing the active ingredients) over a short period of time in the upper part of the intestine, whereas larger granules are digested by lipases more slowly and the release of active ingredients occurs over a longer period of time with respect to the smaller granules and more distal position along the intestinal tract.

TABLE A

| | 50-250 | 250 ÷ 400 | 400 ÷ 500 | 500 ÷ 1000 | 1000 ÷ 1500 | 1500 ÷ 2000 | 2000 ÷ 2500 |
|---|---|---|---|---|---|---|---|
| | um | um | um | um | um | um | um |
| Aquatic | 15% | 50% | 25% | 10% | | | |
| species | | | | | | | |
| Poultry | 5% | | | 50% | 40% | 4.5% | 0.5% |
| species | | | | | | | |

To define the particle size percentage distribution of a batch of the composition of the present invention, instruments and methodologies known to the man skilled in the art can be used for particle size analysis. For example, within the scope of the present invention, one of the following two methods can be used to define said particle size distribution percentage: particle size analysis using certified sieves or particle size analysis using laser diffraction.

The analysis by means of certified sieves (i.e. perforated plates made of stainless steel) is carried out, for example, by means of a vibrating platform with n sieves assembled one over the other in a sieve holder container arranged above the vibrating platform (for example, frequency of about 3000 cycles/min). Each sieve in the sieve holder container has a different size (for example sieves from 250 μm to 2500 μm) and said sieves are positioned one over the other so that the larger sieves are arranged in the upper part of the container and the smaller sieves in the lower part of the container. The container is vibrated and a certain amount of a powder or granules is poured onto the upper sieve: the particles passing through the upper sieves reach the lower sieves or beyond. The operation ends when no evident separation occurs anymore. Stopping the powder on a sieve of a certain size determines its particle size. The sieves are quality certified: the certificate of conformity certifies that the mesh, materials used, dimensions and production process comply with the requirements.

The analysis of the size of solid particles—using the laser diffraction technique—is based on the principle that the particles illuminated by a laser beam diffuse the light at an angle related to the size thereof (the angle increases as the particle size decreases). The average diameter is determined based on the surface/volume ratio, using the parameter D (De Brouckere mean diameter-equation). The dimensional distribution is identified by the following parameters: D (0.1), D (0.5), D (0.9), which represent the cumulative distribution diameters of 10%, 50% and 90% of the total particles.

Said (iii) at least one additive and/or excipient, optionally comprised in the composition of the invention together with (i) and (ii) according to both said first embodiment (FR-I) and said second embodiment (FR-II), is a substance devoid of therapeutic activity suitable for pharmaceutical or food purposes and it can be any ancillary substance known to the man skilled in the art such as, by way of non-limiting example, diluents, solvents (including water, glycerine, ethyl alcohol), solubilisers, thickeners, sweeteners, flavour enhancers, dies, lubricants, surfactants, antimicrobials, antioxidants, preservatives, pH stabilising buffers and mixtures thereof. Non-limiting examples of such ancillary substances are maltodextrins, phosphate buffers, bases such as sodium hydroxide, xanthan gum, guar gum, fructose, natural or artificial flavours. Preferably, said (iii) at least one additive and/or excipient, optionally comprised in the composition of the invention together with (i) and (ii), comprises a coating additive or a mixture of coating additives which are used to increase the viscosity of the lipid matrix and which decrease the permeability thereof and they are selected from group F comprising or, alternatively, consisting of: fumed silica, calcium stearate, magnesium stearate calcium sulfate, precipitated silica, calcium silicate, aluminium silicate, hydrophobic silica and mixtures thereof; preferably fumed silica, calcium sulfate, precipitated silica, calcium silicate, aluminium silicate, hydrophobic silica and mixtures thereof.

Preferably, said (iii) at least one additive and/or excipient or a mixture of additives and/or excipients, preferably comprising at least one of said coating additives selected from group F, is comprised in the composition of the invention at a percentage by weight comprised in the range from 0.1% to 30% (for example 0.5%, 2%, 4%, 6%, 8%, 15% o 25%) with respect to the total weight of the composition of the invention, preferably from 1% to 20%, more preferably from 5% to 10%.

For example, the composition of the invention may comprise (according to FR-II): said (i) mixture of active components comprising or, alternatively, consisting of at least one at least one amino acid and at least one phytocompound derivative, said (ii) controlled release lipid matrix and, optionally, said (iii) at least one coating additive selected from group F, wherein said composition comprises said (i), (ii) and, optionally, (iii) in the following percentages by weight with respect to the total weight of the composition: said (i) from 1% to 90% or 89.9% (for example 5%, 20%, 25%, 30%, 35%, 40%, 50%, 55% or 65%), said (ii) from 10% to 80% (for example 15%, 20%, 25%, 35%, 50% or 65%) and said (iii) from 0.1% to 30%; preferably said (i) from 5% to 50%, said (ii) from 30% to 70%, and said (iii) from 1% to 20%; more preferably said (i) from 15% to 40%, said (ii) from 40% to 60%, and said (iii) from 5% to 10%.

In the context of the present invention, the term “phytocompound derivative (botanical)” is used to indicate a chemical compound generally produced by plants as a secondary metabolite, which can also be chemically synthesised in a laboratory. When extracted from plants, it can be obtained as a powder extract of various parts of the plant such as stem, roots, seeds (dry extract), or as a liquid extract (essential oils) or semi-solid extract (oleoresins). Said phytocompound derivatives (from (a) to (g)) are prepared according to methods and apparatus known to the man skilled in the art, such as for example aqueous or hydroalcoholic extractions from plants (dry extract) or from essential oils, distillations, enzymatic or microbiological processes from material of plant origin in the raw state or after the transformation thereof (by drying, roasting, fermentation, etc.), or chemical synthesis.

Thymol (IUPAC name: 2-isopropyl-5-methylphenol or 3-Hydroxy-4-isopropyltoluene) is a monoterpenoid phenol present in abundant quantities in plants of the genus Thymus, from which it takes its name and to which it contributes to the creation of aroma, together with other molecules such as carvacrol.

Carvacrol (IUPAC name 5-isopropyl-2-methylphenol) or cymophenol is a monoterpenoid phenol (regioisomer of thymol) present in the essential oil of oregano and thymus.

Eugenol (IUPAC name 2-methoxy-4-(prop-2-en-1-yl)-phenol) is a hydroxylated aromatic compound which is extracted from some essential oils, in particular clove oil and from cinnamon.

Capsaicin (IUPAC name (E)-N-(4-hydroxy-3-methoxybenzyl)-8-methylnon-6-enamide) (also called capsicine or capseicin) is an alkaloid present, at various concentrations, in plants of the genus Capsicum (for example in hot chili pepper).

Tannins are a class of compounds (polyphenols) contained in various plants with properties similar to those of tannic acid (IUPAC name: 2,3-dihydroxy-5-({[(2R,3R,4S,5R,6R)-3,4,5,6-tetrakis({3,4-dihydroxy-5-[(3,4,5-trihydroxyphenyl)carbonyloxy]phenyl}carbonyloxy)oxan-2-yl]methoxy}carbonyl)phenyl 3,4,5-trihydroxybenzoate. Tannins are common in vascular plants the sweet chestnut (Castanea sativa) which contains, in the tissues thereof, about 7% of the total, being the richest in tannins. The families of plants most known for the presence of tannins are: Aceraceae, Actinidiaceae, Anacardiaceae, Bixaceae, Burseraceae, Combretaceae, Dipterocarpaceae, Ericaceae, Grossulariaceae, Myricaceae among dicotyledons and Najadaceae and Typhaceae among monocotyledons.

Verbascoside (molecular formula C29H36O15) is a phenylpropanoid which can be obtained by extraction from plants of the order of the Lamiales (family of Scrophulariaceae, such as Verbasicum phlomoides and Verbasum mallophorum) but also in species of the orders of Asterales, Cucurbitales and Magnoliales, or produced in plant cell cultures of Leucosceptrum sp (Lamiaceae) and Syringa sp (Oleaceae).

Saponins (or saponosides) are terpene glycosides of plant origin. They are present in two main classes of the plant kingdom, the Magnoliopsida (dicotyledons) and Liliopsida (monocotyledons).

According to an aspect of said second embodiment of the invention (FR-II), in the composition of the invention (i) (such as (1.1) and (i.2)), (ii) and, optionally, (iii), besides said (i.1) at least one amino acid and said (i.2) at least one phytocompound derivative (according to any one of the described aspects), said (i) mixture of active components further comprises at least one further first active component selected from group C (i.e. ornithine, alpha-ketoglutaric acid, a protease and a polyamine), preferably a protease, more preferably a cysteine-protease selected from the group comprising or, alternatively, consisting of bromelain, papain, ficain and mixtures thereof.

In preferred examples of the composition of the invention according to said second embodiment (FR-11), said composition comprises: said (i) mixture of active components, said (ii) lipid matrix comprises or, alternatively, consists of: a triglyceride or a fatty acid or a wax and a mixture thereof (as defined in the context of the present invention) preferably rapeseed oil, palm oil and soybean oil, and, optionally, said (iii) at least one additive and/or excipient,

wherein said (I) mixture of active components (comprising or, alternatively, consisting of (i.1) at least one amino acid and (i.2) at least one phytocompound derivative) is selected from the group comprising or, alternatively, consisting of:

FR-II-1: lysine and thymol, lysine and carvacrol, lysine and eugenol, lysine and capsaicin, lysine and tannins, lysine and verbascoside, lysine and saponins,

lysine and thymol and carvacrol, lysine and thymol and eugenol, lysine and thymol and capsaicin, lysine and thymol and tannins, lysine and thymol and verbascoside, lysine and thymol and saponins, lysine and carvacrol and eugenol, lysine and carvacrol and capsaicin, lysine and carvacrol and tannins, lysine and carvacrol and verbascoside, lysine and carvacrol and saponins; or

FR-II-2: methionine and thymol, methionine and carvacrol, methionine and eugenol, methionine and capsaicin, methionine and tannins, methionine and verbascoside, methionine and saponins, methionine and thymol and carvacrol, methionine and thymol and eugenol, methionine and thymol and capsaicin, methionine and thymol and tannins, methionine and thymol and verbascoside, methionine and thymol and saponins,

methionine and carvacrol and eugenol, methionine and carvacrol and capsaicin, methionine and carvacrol and tannins, methionine and carvacrol and verbascoside, methionine and carvacrol and saponins; or

FR-II-3: threonine and thymol, threonine and carvacrol, threonine and eugenol, threonine and capsaicin, threonine and tannins, threonine and verbascoside, threonine and saponins,

threonine and thymol and carvacrol, threonine and thymol and eugenol, threonine and thymol and capsaicin, threonine and thymol and tannins, threonine and thymol and verbascoside, threonine and thymol and saponins,

threonine and carvacrol and eugenol, threonine and carvacrol and capsaicin, threonine and carvacrol and tannins, threonine and carvacrol and verbascoside, threonine and carvacrol and saponins; or

FR-II-4: arginine and thymol, arginine and carvacrol, arginine and eugenol, arginine and capsaicin, arginine and tannins, arginine and verbascoside, arginine and saponins,

arginine and thymol and carvacrol, arginine and thymol and eugenol, arginine and thymol and capsaicin, arginine and thymol and tannins, arginine and thymol and verbascoside, arginine and thymol and saponins,

arginine and carvacrol and eugenol, arginine and carvacrol and capsaicin, arginine and carvacrol and tannins, arginine and carvacrol and verbascoside, arginine and carvacrol and saponins; or

FR-II-5: lysine and methionine and thymol, lysine and methionine and carvacrol, lysine and methionine and eugenol, lysine and methionine and capsaicin, lysine and methionine and tannins, lysine and methionine and verbascoside, lysine and methionine and saponins,

lysine and methionine and thymol and carvacrol, lysine and methionine and thymol and eugenol, lysine and methionine and thymol and capsaicin, lysine and methionine and thymol and tannins, lysine and methionine and thymol and verbascoside, lysine and methionine and thymol and saponins,

lysine and methionine and carvacrol and eugenol, lysine and methionine and carvacrol and capsaicin, lysine and methionine and carvacrol and tannins, lysine and methionine and carvacrol and verbascoside, lysine and methionine and carvacrol and saponins; or

FR-II-6: lysine and methionine and threonine and thymol, lysine and methionine and threonine and carvacrol, lysine and methionine and threonine and eugenol, lysine and methionine and threonine and capsaicin, lysine and methionine and threonine and tannins, lysine and methionine and threonine and verbascoside, lysine and methionine and threonine and saponins,

lysine and methionine and threonine and thymol and carvacrol, lysine and methionine and threonine and thymol and eugenol, lysine and methionine and threonine and thymol and capsaicin, lysine and methionine and threonine and thymol and tannins, lysine and methionine and threonine and thymol and verbascoside, lysine and methionine and threonine and thymol and saponins,

lysine and methionine and threonine and carvacrol and eugenol, lysine and methionine and threonine and carvacrol and capsaicin, lysine and methionine and threonine and carvacrol and tannins, lysine and methionine and threonine and carvacrol and verbascoside, lysine and methionine and threonine and carvacrol and saponins; or

FR-II-7: lysine and methionine and arginine and thymol, lysine and methionine and arginine and carvacrol, lysine and methionine and arginine and eugenol, lysine and methionine and arginine and capsaicin, lysine and methionine and arginine and tannins, lysine and methionine and arginine and verbascoside, lysine and methionine and arginine and saponins,

lysine and methionine and arginine and thymol and carvacrol, lysine and methionine and arginine and thymol and eugenol, lysine and methionine and arginine and thymol and capsaicin, lysine and methionine and arginine and thymol and tannins, lysine and methionine and arginine and thymol and verbascoside, lysine and methionine and arginine and thymol and saponins,

lysine and methionine and arginine and carvacrol and eugenol, lysine and methionine and arginine and carvacrol and capsaicin, lysine and methionine and arginine and carvacrol and tannins, lysine and methionine and arginine and carvacrol and verbascoside, lysine and methionine and arginine and carvacrol and saponins; or

FR-II-8: lysine and tryptophan and thymol, lysine and tryptophan and carvacrol, lysine and tryptophan and eugenol, lysine and tryptophan and capsaicin, lysine and tryptophan and tannins, lysine and tryptophan and verbascoside, lysine and tryptophan and saponins, lysine and tryptophan and thymol and carvacrol;

methionine and tryptophan and thymol, methionine and tryptophan and carvacrol, methionine and tryptophan and eugenol, methionine and tryptophan and capsaicin, methionine and tryptophan and tannins, methionine and tryptophan and verbascoside, methionine and tryptophan and saponins, methionine and tryptophan and thymol and carvacrol;

threonine and tryptophan and thymol, threonine and tryptophan and carvacrol, threonine and tryptophan and eugenol, threonine and tryptophan and capsaicin, threonine and tryptophan and tannins, threonine and tryptophan and verbascoside, threonine and tryptophan and saponins, threonine and tryptophan and thymol and carvacrol;

arginine and tryptophan and thymol, arginine and tryptophan and carvacrol, arginine and tryptophan and eugenol, arginine and tryptophan and capsaicin, arginine and tryptophan and tannins, arginine and tryptophan and verbascoside, arginine and tryptophan and saponins, arginine and tryptophan and thymol and carvacrol;

lysine and methionine and tryptophan and thymol, lysine and methionine and tryptophan and carvacrol, lysine and methionine and tryptophan and eugenol, lysine and methionine and tryptophan and capsaicin, lysine and methionine and tryptophan and tannins, lysine and methionine and tryptophan and verbascoside, lysine and methionine and tryptophan and saponins, lysine and methionine and tryptophan and thymol and carvacrol; or

FR-II-9: lysine and histidine and thymol, lysine and histidine and carvacrol, lysine and histidine and eugenol, lysine and histidine and capsaicin, lysine and histidine and tannins, lysine and histidine and verbascoside, lysine and histidine and saponins, lysine and histidine and thymol and carvacrol;

methionine and histidine and thymol, methionine and histidine and carvacrol, methionine and histidine and eugenol, methionine and histidine and capsaicin, methionine and histidine and tannins, methionine and histidine and verbascoside, methionine and histidine and saponins, methionine and histidine and thymol and carvacrol;

threonine and histidine and thymol, threonine and histidine and carvacrol, threonine and histidine and eugenol, threonine and histidine and capsaicin, threonine and histidine and tannins, threonine and histidine and verbascoside, threonine and histidine and saponins, threonine and histidine and thymol and carvacrol;

arginine and histidine and thymol, arginine and histidine and carvacrol, arginine and histidine and eugenol, arginine and histidine and capsaicin, arginine and histidine and tannins, arginine and histidine and verbascoside, arginine and histidine and saponins, arginine and histidine and thymol and carvacrol;

lysine and methionine and histidine and thymol, lysine and methionine and histidine and carvacrol, lysine and methionine and histidine and eugenol, lysine and methionine and histidine and capsaicin, lysine and methionine and histidine and tannins, lysine and methionine and histidine and verbascoside, lysine and methionine and histidine and saponins, lysine and methionine and histidine and thymol and carvacrol; or

FR-II-10: lysine and thymol and valine and/or isoleucine, lysine and carvacrol and valine and/or isoleucine, lysine and histidine and eugenol, lysine and capsaicin and valine and/or isoleucine, lysine and tannins and valine and/or isoleucine, lysine and verbascoside and valine and/or isoleucine, lysine and saponins and valine and/or isoleucine, lysine and thymol and carvacrol and valine and/or isoleucine;

methionine and thymol and valine and/or isoleucine, methionine and carvacrol and valine and/or isoleucine, methionine and histidine and eugenol, methionine and capsaicin and valine and/or isoleucine, methionine and tannins and valine and/or isoleucine, methionine and verbascoside and valine and/or isoleucine, methionine and saponins and valine and/or isoleucine, methionine and thymol and carvacrol and valine and/or isoleucine;

threonine and thymol and valine and/or isoleucine, threonine and carvacrol and valine and/or isoleucine, threonine and eugenol and valine and/or isoleucine, threonine and capsaicin and valine and/or isoleucine, threonine and tannins and valine and/or isoleucine, threonine and verbascoside and valine and/or isoleucine, threonine and saponins and valine and/or isoleucine, threonine and thymol and carvacrol and valine and/or isoleucine;

arginine and thymol and valine and/or isoleucine, arginine and carvacrol and valine and/or isoleucine, arginine and eugenol and valine and/or isoleucine, arginine and capsaicin and valine and/or isoleucine, arginine and tannins and valine and/or isoleucine, arginine and verbascoside and valine and/or isoleucine, arginine and saponins and valine and/or isoleucine, arginine and thymol and carvacrol and valine and/or isoleucine;

lysine and methionine and thymol and valine and/or isoleucine, lysine and methionine and carvacrol and valine and/or isoleucine, lysine and methionine and eugenol and valine and/or isoleucine, lysine and methionine and capsaicin and valine and/or isoleucine, lysine and methionine and tannins and valine and/or isoleucine, lysine and methionine and verbascoside and valine and/or isoleucine, lysine and methionine and saponins and valine and/or isoleucine, lysine and methionine and thymol and carvacrol and valine and/or isoleucine.

In preferred examples of the composition of the invention according to FR-II, said composition comprises: said (i) mixture of active components, said (ii) lipid matrix comprises or, alternatively, consists of rapeseed oil and, optionally, said (iii) at least one additive and/or excipient (preferably coating additives), wherein said (i) mixture of active components is selected from the group comprising or, alternatively, consisting of what is listed in FR-II-1, FR-II-2, FR-II-3, FR-II-4, FR-II-5, FR-II-6, FR-II-7, FR-II-8, FR-II-9 and FR-II-10.

In preferred examples of the composition of the invention according to FR-II, said composition comprises: said (i) mixture of active components, said (ii) lipid matrix comprises or, alternatively, consists of palm oil and, optionally, said (iii) at least one additive and/or excipient (preferably coating additives), wherein said (i) mixture of active components is selected from the group comprising or, alternatively, consisting of what is listed in FR-II-1, FR-II-2, FR-II-3, FR-II-4, FR-II-5, FR-II-6, FR-II-7, FR-II-8, FR-II-9 and FR-II-10.

In preferred examples of the composition of the invention according to FR-II, said composition comprises: said (i) mixture of active components, said (ii) lipid matrix comprises or, alternatively, consists of soybean oil and, optionally, said (iii) at least one additive and/or excipient (preferably coating additives), wherein said (i) mixture of active components is selected from the group comprising or, alternatively, consisting of what is listed in FR-II-1, FR-II-2, FR-II-3, FR-II-4, FR-II-5, FR-II-6, FR-II-7, FR-II-8, FR-II-9 and FR-II-10.

Preferably, in said embodiments of the invention comprising from FR-II-1 to FR-II-10, said (i) mixture comprises saponins when said non-mammalian monogastric animal is a poultry animal.

Preferably, in said embodiments of the invention comprising from FR-II-1 to FR-II-10, said (i) mixture comprises arginine or histidine when said non-mammalian monogastric animal is a fish or crustacean.

In said second embodiment (FR-II) of the composition of the present invention, preferably, in said (i) mixture of active components, the [(i.1) at least one amino acid: (i.2) at least one phytocompound derivative] by weight ratio is comprised in the range from 1:10 to 10:1, preferably from 10:1 to 10:5, more preferably from 10:1 to 10:3.

The feed or feed additives of the present invention, comprising the composition of the present invention according both to said first embodiment (FR-I) and said second embodiment (FR-II), may be in solid form, such as granules, flakes, powder, in semi-solid form or in liquid form, such as such as suspensions, dispersions, solutions, emulsions. Besides the composition of the invention, said feed further comprises appropriate nutrients depending on the type of animal (such as poultry or fish) based on the knowledge of the man skilled in the art.

Unless otherwise specified, the expression composition comprising a component at an amount “comprised in a range from x to y” is used to indicate that said component may be present in the composition at all the amounts present in said range, even if not stated, extremes of the range included.

Preferred aspects)(FR-I-n° of said first embodiment of the invention (FR-I) are reported below: FR-I-1. A composition comprising (i), (H) and, optionally, (iii) for use in a method for treating the amino acid supply in a non-mammal monogastric animal, wherein

(i) is at least one active component or a mixture of active components comprising, or alternatively, consisting of at least one amino acid or an analogue or an acceptable pharmaceutical or food grade salt thereof;

(ii) is a lipid matrix embedding said (i) at least one active component or mixture of active components, wherein said lipid matrix comprising or, alternatively, consisting of at least one saturated or unsaturated, free or esterified fatty acid having a number of carbon atoms comprised in the range C10-C30, and/or at least one triglyceride having saturated or unsaturated fatty acid chains, having a number of carbon atoms comprised in the range C6-C30 and/or at least one wax having a number of carbon atoms comprised in the range C16-C36; and

(iii) is at least one acceptable pharmaceutical or food grade additive and/or excipient;

wherein said (ii) lipid matrix provides gastroprotection, in case of an animal with a stomach, and a controlled release in the intestine and/or in the hepatopancreas of said (i) at least one or mixture of active components, guaranteeing a constant bioavailability within a period of time comprised in the range from 2 hours to 24 hours;

and wherein said (ii) lipid matrix decreases the leaching of said (i) at least one or mixture of active components in the aquatic environment.

FR-I-2. The composition for use according to FR-I-1, wherein said at least one amino acid or analogue is selected from the group comprising or, alternatively, consisting of: methionine, lysine, valine, isoleucine, tryptophan, histidine, leucine, phenylalanine, threonine, arginine, cysteine, tyrosine, glutamine, proline, hydroxyproline, glycine, and taurine and mixtures thereof; preferably methionine, lysine and threonine.

FR-I-3. The composition for use according to FR-I-1 or FR-I-2, wherein said at least one amino acid is a mixture of amino acids selected from the group of mixtures comprising or, alternatively, consisting of: methionine and lysine; methionine and threonine; lysine and threonine; methionine and glutamine; lysine and glutamine; threonine and glutamine; tryptophan and glutamine; valine and glutamine; leucine and glutamine; isoleucine and glutamine; methionine, lysine and threonine; methionine, lysine and cysteine; valine, isoleucine and tryptophan; valine, isoleucine and threonine; methionine, valine, isoleucine and tryptophan; methionine, lysine, valine, isoleucine and tryptophan methionine, lysine, cysteine and valine; methionine, lysine, cysteine and isoleucine; methionine, lysine, cysteine, tryptophan; methionine, lysine, cysteine, valine and isoleucine; methionine, lysine, cysteine, valine and tryptophan; methionine, lysine, cysteine, isoleucine and tryptophan; methionine, lysine, cysteine, valine, isoleucine and tryptophan; leucine, valine and isoleucine; methionine, leucine, valine and isoleucine; methionine, lysine, leucine, valine and isoleucine; methionine, lysine, cysteine, leucine, valine and isoleucine; preferably methionine and lysine; methionine, lysine and threonine; methionine, lysine and cysteine; leucine, valine and isoleucine; valine, isoleucine and tryptophan.

FR-I-4. The composition for use according to any one of the preceding FRs-I, wherein said (i) mixture of active components further comprises at least one first further active component selected from the group comprising or, alternatively consisting of: a non-proteinogenic amino acid selected from ornithine, alpha-ketoglutaric acid and mixtures thereof; a protease selected from bromelain, papain, ficain and mixtures thereof; a polyamine selected from putrescine, spermine, spermidine and mixtures thereof.

FR-I-5. The composition for use according to any one of the preceding FRs-I, wherein said (i) mixture of active components further comprises at least one second further active component selected from the group comprising or, alternatively consisting of: at least one vitamin, preferably a vitamin of group B, at least one organic or inorganic acid, at least one mineral salt, preferably an organic or inorganic salt of a Fe, Se, Mg, Ca, K, Zn or Cu cation, at least one antioxidant, at least one probiotic bacterial strain, at least one prebiotic, at least one enzyme and mixtures thereof.

FR-I-6. The composition for use according to any one of the preceding FRs-I, wherein said (iii) at least one additive and/or excipient comprises at least one coating additive selected from the group comprising or, alternatively, consisting of: fumed silica, calcium stearate, magnesium stearate, calcium sulfate, precipitated silica, calcium silicate, aluminium silicate, hydrophobic silica.

FR-I-7. Use of the composition according to any one of FRs-I from 1 to 6 for preparing an animal feed or feed additive for a non-mammalian monogastric animal.

FR-I-8. An feed or feed additive for a non-mammalian monogastric animal comprising or alternatively consisting of the composition according to any one of FRs-I from 1 to 6.

FR-I-9. The feed or feed additive according to FR-I-8, wherein said non-mammalian monogastric animal is an animal of the poultry species or of the aquatic species or a reptile, preferably chicken or fowls, turkey, guinea fowl, pheasant, peacock, partridge, quail, dove, turtle dove, goose, common duck, mute duck, fish and crustaceans.

FR-I-10. The feed or feed additive according to FR-I-8 or FR-I-9, wherein said feed or feed additive has the function of providing the required daily average amino acid supply and/or of increasing and/or accelerating the development of the body mass and/or decreasing the excretion nitrogenated compounds regarding said non-mammalian monogastric animal

Experimental Part I

A method for measuring the plasma bioavailability of amino acids in a non-mammalian monogastric animal following the administration of a composition according to the present invention (comprising at least one amino acid, at least one phytocompound and a lipid matrix) consists of:
administer the following diets to 3 experimental groups of an animal species under study (for example, chicken or fish):

group 1. a control diet (for example soy-based),

group 2. a diet added with a comparison composition: composition comprising amino acids and phytocompound derivatives in the absence of a lipid matrix (non-embedded active components), and

group 3. a diet added with a composition according to the invention: composition comprising amino acids and phytocompound derivatives in the presence of a lipid matrix (embedded active components)
collect blood samples from the animals under study and obtain the plasma fraction. The samples are collected at different time-points after the administration of the diets (from 10 minutes up to 360 minutes after the administration) and the presence of one or more amino acids in the obtained plasma fractions is evaluated by means of the LC/MS-MS (Liquid Chromatography with tandem Mass Spectrometry) plasma amino acid assay method.

Experimental Part II

Table 2 shows the values of the experimental study which analysed the release of phytocompound derivatives embedded in a lipid matrix in the form of granules (composition according to the invention). As the data show, the release is a function of the time and size of the granules, the larger the granule size, the slower the release of the active ingredient.

The data were obtained by incubating 1 gram of granules of different sizes in a buffer simulating the intestinal pH conditions. At each time point (1 hour, 2 hours, 4 hours) the phytocompound derivatives still present in the granules were quantified (by means of HPLC), and the release percentages were calculated by difference. The experiment was triplicated.

TABLE 2

| | | | |
|---|---|---|---|
| | Fraction at | Fraction at | Fraction at |
| Particle size | 1 hour | 2 hours | 3 hours |

| | | | | |
|---|---|---|---|---|
| 2000-2500 | um | 0 | 0 | 0 |
| 1500-2000 | um | 0 | 17% | 8% |
| 1000-1500 | um | 0 | 18% | 3% |
| 500-1000 | um | 0 | 15% | 20% |
| 50-500 | um | 26% | 36% | 48% |

### Examples

Representative examples of compositions of the invention according to the second embodiment (FR-II) are shown in Table 3

TABLE 3
(%): weight/weight composition. AA: Amino acid. der-FT: phytocompound derivative [(a) thymol,
(b) carvacrol, (c) eugenol, (d) capsaicin, (e) tannins, (f) verbascosde, (g) saponins]. Add: additive.

| | | | | der- | | | Matrix oil |
|---|---|---|---|---|---|---|---|
| | AA1 (%) | AA2 (%) | AA3 (%) | FT1(%) | der-FT2(%) | Add (%) | (%) |
| Comp 1 | Lys (18%) | Met (15%) | — | a (5%) | b (2%) | (5%) | rapeseed |
| Comp 2 | Lys (10%) | Met (5%) | Thr (5%) | a (10%) | c (5%) | (3%) | rapeseed |
| Comp 3 | Lys (15%) | Val (10%) | IsoLeu (10%) | b (12%) | c (2%) | (1%) | palm (50%) |
| Comp 4 | Thr (10%) | Met (5%) | Trp (8%) | b (4%) | d (10%) | (3%) | palm (60%) |
| Comp 5 | Thr (10%) | Val (5%) | IsoLeu (5%) | d (10%) | g (6%) | (10%) | soy (54%) |
| Comp 6 | Arg (10%) | Met (15%) | — | c (15%) | f (2%) | (3%) | soy (55%) |
| Comp 7 | Lys (10%) | Met (15%) | His (5%) | a (8%) | e (2%) | (15%) | rapeseed (45%) |

1. A solid composition for the treatment of deficiency of at least one amino acid in a non-mammalian monogastric animal,
wherein said non-mammalian monogastric animal is a poultry animal or a fish or a crustacean, and
wherein said composition comprises
a mixture of active components comprising:
at least one amino acid or an acceptable pharmaceutical or food grade salt thereof, wherein said amino acid is selected from the group consisting of lysine, methionine, tryptophan, threonine, valine, isoleucine, arginine, histidine and mixtures thereof; and
at least one phytocompound derivative selected from the group consisting of thymol, carvacrol, eugenol, capsaicin, tannins, verbascoside, saponins and mixtures thereof;
wherein said composition further comprises
a lipid matrix embedding said mixture of active components, wherein said lipid matrix comprises:
at least one saturated or unsaturated, free or esterified fatty acid having a number of carbon atoms in the range C10-C30, and/or
at least one triglyceride having saturated or unsaturated fatty acid chains, having a number of carbons in the range C6-C30 and/or
at least one wax having a number of carbon atoms in the range C16-C36;
wherein said composition is formulated in the form of granules having the following particle size distribution percentage with respect to 100 granules:
when said animal is a fish or a crustacean: from 10% to 20% of granules having an average particle size from 50 μm to 250 μm, from 45% to 55% of granules having an average particle size from 250 μm to 400 μm, from 20% to 30% of granules having an average particle size from 400 μm to 500 μm, from 5% to 15% of granules having an average particle size from 500 μm to 2500 μm;
when said animal is a poultry animal: from 1% to 10% of granules having an average particle size from 50 μm to 500 μm, from 45% to 55% of granules having an average particle size from 500 μm to 1000 μm, from 35% to 45% of granules having an average particle size from 1000 μm to 1500 μm, from 1% to 9% of granules having an average particle size from 1500 μm to 2000 μm, from 0.1% to 1% of granules having an average particle size from 2000 μm to 2500 μm;
wherein said composition is formulated for administration through oral route,
wherein said lipid matrix is capable of providing gastroprotection of said at least one amino acid and said at least one phytocompound derivative,
wherein said lipid matrix is capable of providing a controlled release of said at least one amino acid and said at least one phytocompound derivative within a time range comprised from 30 minutes to 8 hours in the intestinal tract, and
wherein said lipid matrix is capable of providing a plasmatic bioavailability of said at least one amino acid in a constant percentage over a period of time comprised from 2 hours to 24 hours.

2. The composition according to claim 1, wherein said lipid matrix is selected from the group consisting of: rapeseed oil, palm oil, soybean oil and a mixture thereof.

3. The composition according to claim 1,
wherein said mixture of active components comprises lysine and said at least one phytocompound derivative, and
wherein said lipid matrix comprises rapeseed oil or palm oil or soybean oil or a mixture thereof.

4. The composition according to claim 1,
wherein said mixture of active components comprises methionine and said at least one phytocompound derivative, and
wherein said lipid matrix comprises rapeseed oil or palm oil or soybean oil or a mixture thereof.

5. The composition according to claim 1,
wherein said mixture of active components comprises threonine and said at least one phytocompound derivative, and
wherein said lipid matrix comprises rapeseed oil or palm oil or soybean oil or a mixture thereof.

6. The composition according to claim 1,
wherein said mixture of active components comprises arginine and said at least one phytocompound derivative, and
wherein said lipid matrix comprises rapeseed oil or palm oil or soybean oil or a mixture thereof.

7. The composition according to claim 1,
wherein said mixture of active components comprises lysine and methionine and said at least one phytocompound derivative, and
wherein said lipid matrix comprises rapeseed oil or palm oil or soybean oil or a mixture thereof.

8. The composition according to claim 1,
wherein said mixture of active components comprises lysine and methionine and threonine and said at least one phytocompound derivative, and
wherein said lipid matrix comprises rapeseed oil or palm oil or soybean oil or a mixture thereof.

9. The composition according to claim 1,
wherein said mixture of active components comprises lysine and methionine and arginine and said at least one phytocompound derivative, and
wherein said lipid matrix comprises rapeseed oil or palm oil or soybean oil or a mixture thereof.

10. The composition according to claim 1,
wherein said mixture of active components is selected from the group consisting of
lysine and tryptophan and thymol, lysine and tryptophan and carvacrol, lysine and tryptophan and eugenol, lysine and tryptophan and capsaicin, lysine and tryptophan and tannins, lysine and tryptophan and verbascoside, lysine and tryptophan and saponins, lysine and tryptophan and thymol and carvacrol;
methionine and tryptophan and thymol, methionine and tryptophan and carvacrol, methionine and tryptophan and eugenol, methionine and tryptophan and capsaicin, methionine and tryptophan and tannins, methionine and tryptophan and verbascoside, methionine and tryptophan and saponins, methionine and tryptophan and carvacrol;
threonine and tryptophan and thymol, threonine and tryptophan and carvacrol, threonine and tryptophan and eugenol, threonine and tryptophan and capsaicin, threonine and tryptophan and tannins, threonine and tryptophan and verbascoside, threonine and tryptophan and saponins, threonine and tryptophan and thymol and carvacrol;
arginine and tryptophan and thymol, arginine and tryptophan and carvacrol, arginine and tryptophan and eugenol, arginine and tryptophan and capsaicin, arginine and tryptophan and tannins, arginine and tryptophan and verbascoside, arginine and tryptophan and saponins, arginine and tryptophan and thymol and carvacrol;
lysine and methionine and tryptophan and thymol, lysine and methionine and tryptophan and carvacrol, lysine and methionine and tryptophan and eugenol, lysine and methionine and tryptophan and capsaicin, lysine and methionine and tryptophan and tannins, lysine and methionine and tryptophan and verbascoside, lysine and methionine and tryptophan and saponins, lysine and methionine and tryptophan and thymol and carvacrol; and
wherein said lipid matrix comprises rapeseed oil or palm oil or soybean oil or a mixture thereof.

11. The composition according to claim 1,
wherein said mixture of active components is selected from the group consisting of
lysine and histidine and thymol, lysine and histidine and carvacrol, lysine and histidine and eugenol, lysine and histidine and capsaicin, lysine and histidine and tannins, lysine and histidine and verbascoside, lysine and histidine and saponins, lysine and histidine and thymol and carvacrol;
methionine and histidine and thymol, methionine and histidine and carvacrol, methionine and histidine and eugenol, methionine and histidine and capsaicin, methionine and histidine and tannins, methionine and histidine and verbascoside, methionine and histidine and saponins, methionine and histidine and thymol and carvacrol;
threonine and histidine and thymol, threonine and histidine and carvacrol, threonine and histidine and eugenol, threonine and histidine and capsaicin, threonine and histidine and tannins, threonine and histidine and verbascoside, threonine and histidine and saponins, threonine and histidine and thymol and carvacrol;
arginine and histidine and thymol, arginine and histidine and carvacrol, arginine and histidine and eugenol, arginine and histidine and capsaicin, arginine and histidine and tannins, arginine and histidine and verbascoside, arginine and histidine and saponins, arginine and histidine and thymol and carvacrol;
lysine and methionine and histidine and thymol, lysine and methionine and histidine and carvacrol, lysine and methionine and histidine and eugenol, lysine and methionine and histidine and capsaicin, lysine and methionine and histidine and tannins, lysine and methionine and histidine and verbascoside, lysine and methionine and histidine and saponins, lysine and methionine and histidine and thymol and carvacrol; and
wherein said lipid matrix comprises rapeseed oil or palm oil or soybean oil or a mixture thereof.

12. The composition according to claim 1,
wherein said mixture of active components is selected from the group consisting of
lysine and thymol and valine and/or isoleucine, lysine and carvacrol and valine and/or isoleucine, lysine and histidine and eugenol, lysine and capsaicin and valine and/or isoleucine, lysine and tannins and valine and/or isoleucine, lysine and verbascoside and valine and/or isoleucine, lysine and saponins and valine and/or isoleucine, lysine and thymol and carvacrol and valine and/or isoleucine;
methionine and thymol and valine and/or isoleucine, methionine and carvacrol and valine and/or isoleucine, methionine and eugenol and valine and/or isoleucine, methionine and capsaicin and valine and/or isoleucine, methionine and tannins and valine and/or isoleucine, methionine and verbascoside and valine and/or isoleucine, methionine and saponins and valine and/or isoleucine, methionine and thymol and carvacrol and valine and/or isoleucine;
threonine and thymol and valine and/or isoleucine, threonine and carvacrol and valine and/or isoleucine, threonine and eugenol and valine and/or isoleucine, threonine and capsaicin and valine and/or isoleucine, threonine and tannins and valine and/or isoleucine, threonine and verbascoside and valine and/or isoleucine, threonine and saponins and valine and/or isoleucine, threonine and thymol and carvacrol and valine and/or isoleucine;
arginine and thymol and valine and/or isoleucine, arginine and carvacrol and valine and/or isoleucine, arginine and eugenol and valine and/or isoleucine, arginine and capsaicin and valine and/or isoleucine, arginine and tannins and valine and/or isoleucine, arginine and verbascoside and valine and/or isoleucine, arginine and saponins and valine and/or isoleucine, arginine and thymol and carvacrol and valine and/or isoleucine;
lysine and methionine and thymol and valine and/or isoleucine, lysine and methionine and carvacrol and valine and/or isoleucine, lysine and methionine and eugenol and valine and/or isoleucine, lysine and methionine and capsaicin and valine and/or isoleucine, lysine and methionine and tannins and valine and/or isoleucine, lysine and methionine and verbascoside and valine and/or isoleucine, lysine and methionine and saponins and valine and/or isoleucine, lysine and methionine and thymol and carvacrol and valine and/or isoleucine; and
wherein said lipid matrix comprises rapeseed oil or palm oil or soybean oil or a mixture thereof.

13. The composition according to claim 1, wherein said composition further comprises at least one acceptable pharmaceutical or food grade additive and/or excipient,
wherein said at least one additive and/or excipient comprises at least one coating additive selected from the group consisting of fumed silica, calcium stearate, magnesium stearate, calcium sulfate, precipitated silica, calcium silicate, aluminium silicate, hydrophobic silica.

14. The composition according to claim 1, wherein said composition comprises at a percentage by weight with respect to the total weight of the composition:
said mixture of active components from 5% to 50%, said lipid matrix from 30% to 70%.

15. The composition according to claim 1, wherein said poultry animal is selected from the group consisting of chicken, laying hen and turkey, and/or said fish is a fish that can be farmed in fresh water or that can be farmed in salty water;
and/or said crustacean is a crustacean that can be farmed, preferably a prawn.

16. An animal feed or feed additive for a non-mammalian monogastric animal comprising a composition according to claim 1,
wherein said non-mammalian monogastric animal is a poultry animal or a fish or a crustacean, preferably chicken, laying hen, turkey, fish that can be farmed in freshwater or fish that can be farmed in salty water, or crustacean that can be farmed.

17. The composition according to claim 1, wherein said mixture of active components is selected from the group consisting of
lysine and thymol, lysine and carvacrol, lysine and eugenol, lysine and capsaicin, lysine and tannins, lysine and verbascoside, lysine and saponins,
lysine and thymol and carvacrol, lysine and thymol and eugenol, lysine and thymol and capsaicin, lysine and thymol and tannins, lysine and thymol and verbascoside, lysine and thymol and saponins,
lysine and carvacrol and eugenol, lysine and carvacrol and capsaicin, lysine and carvacrol and tannins, lysine and carvacrol and verbascoside, lysine and carvacrol and saponins, and
wherein said lipid matrix comprises rapeseed oil or palm oil or soybean oil or a mixture thereof.

18. The composition according to claim 1, wherein said mixture of active components is selected from the group consisting of
methionine and thymol, methionine and carvacrol, methionine and eugenol, methionine and capsaicin, methionine and tannins, methionine and verbascoside, methionine and saponins,
methionine and thymol and carvacrol, methionine and thymol and eugenol, methionine and thymol and capsaicin, methionine and thymol and tannins, methionine and thymol and verbascoside, methionine and thymol and saponins,
methionine and carvacrol and eugenol, methionine and carvacrol and capsaicin, methionine and carvacrol and tannins, methionine and carvacrol and verbascoside, methionine and carvacrol and saponins, and
wherein said lipid matrix comprises rapeseed oil or palm oil or soybean oil or a mixture thereof.

19. The composition according to claim 13, wherein said composition comprises at a percentage by weight with respect to the total weight of the composition:
said mixture of active components from 5% to 50%, said lipid matrix from 30% to 70%, and, said coating additive from 1% to 20%.

20. A method for treatment of a deficiency of at least one amino acid in a non-mammalian monogastric animal, comprising administering to the monogastric animal the composition according to claim 1.
